(19) United States
(12) Patent Application Publication
(10) Pub. No.: US 2025/0340809 A1
(43) Pub. Date: Nov. 6, 2025

(54) FLEXIBLE BIOPROCESSING VESSEL

(71) Applicant: Global Life Sciences Solutions USA LLC, Marlborough, MA (US)

(72) Inventor: Kevin Lamport, Portsmouth Hampshire (GB)

(73) Assignee: Global Life Sciences Solutions USA LLC, Marlborough, MA (US)

(21) Appl. No.: 18/654,241

(22) Filed: May 3, 2024

(51) Int. Cl. C12M 1/00; C12M 1/12; C12M 3/00

(52) U.S. Cl. CPC C12M 23/26; C12M 23/06; C12M 23/48; C12M 23/52

(57) ABSTRACT

A flexible bioprocessing vessel includes at least six side panels forming sides of the flexible bioprocessing vessel, a top panel adjoining the at least six side panels, the top panel forming a top of the flexible bioprocessing vessel, a bottom panel adjoining the at least six side panels, the bottom panel forming a bottom of the flexible bioprocessing vessel, the at least six side panels, top panel, and bottom panel defining an interior cavity configured for processing a fluid, the at least six side panels joined together via vertical welds. The vessel further includes at least one fluid input and at least one fluid output for adding and removing fluid to and from the interior cavity respectively. Wherein the flexible bioprocessing vessel is configured for use within a rigid support structure having a cylindrical interior.

### BACKGROUND

### Technical Field

Embodiments of the invention relate generally to bioprocessing, and, more particularly, to a flexible bioprocessing vessel.

### Discussion of Art

Mixers and bioreactors are often employed to carry out biochemical and biological processes and/or manipulate liquids and other products of such processes. These devices typically utilize single-use vessels e.g., flexible or collapsible bags that are supported by an outer rigid structure such as a stainless-steel housing/tank. As will be appreciated, use of sterilized single use bags eliminates the time-consuming step of cleaning the tank after each use and reduces the chance of contamination.

In use, a disposable/single-use bag is positioned within the rigid tank and filled with the desired fluid for processing. An impeller assembly that includes a rotating impeller having one or more blades is disposed within the bag and is used to mix the fluid. Existing impeller systems are either top-driven, having a shaft that extends downwardly into the bag, on which one or more impellers are mounted, or bottom-driven, having an impeller disposed in the bottom of the bag that is driven by, for example, a magnetic drive system positioned outside the bag.

Rigid support structures, e.g., stainless steel tanks, may be relatively large, having capacities of 2000 L-3000 L or more. As will be appreciated, 2000 L and 3000 L flexible bags configured for use within such support structures are likewise relatively large and given their size and flexible nature, may be challenging to ship, install and deploy within such structures.

For example, many known bags, particularly larger capacity bags, may require mechanical assistance when being deployed within a rigid base. In particular, a powered mechanical hoist mechanism may be used along with air inflation to guide and raise the vessel to its fully deployed state.

Known bags have hoist points which may assist in raising them into a deployed stated. However, these bags include relatively few hoist points (e.g., 2-4 such points). These attachment points are too few and/or improperly located to prevent or reduce the possibility of wrinkling of the flexible bag during deployment. Such wrinkling is undesirable as it creates localized areas within the bioprocess media that are not uniform with the rest of the bulk fluid, e.g. depleted oxygen zones. When installing bags into support structures, user try to minimize wrinkles and sometimes pull/manipulate bags in an effort to minimize these wrinkles. Such manipulation can increase the chance of accidentally compromising the bag.

Many rigid support structures have cylindrical interiors and are used with cylindrical or semi-cylindrical flexible bags that are manufactured from a relatively small number of flexible panels (e.g., four such panels) that are joined via thermal welding. Despite having relatively few panels, such bags require a large number of welds to manufacture and are generally oversized, e.g., dimensionally larger than necessary to accommodate the maximum fill levels of the bag.

Moreover, the panel configuration and weld location of such bags may result in bags that are folded for shipment in a manner that is relatively space inefficient. As will be appreciated, this may be of particular concern for larger bags, e.g., 2000 L or greater, which are ideally shipped on standard dimension pallets, e.g., 1200 mm×800 mm pallets. Bags that have four panel configurations also may not conform closely to a cylindrical interior of a rigid support structure and may have headspace that is not uniform in curvature or size.

In view of the above, there is a need for a flexible bioprocessing vessel that may be efficiently and economically manufactured, easily shipped using conventional methods, and deployed within cylindrical rigid support structures with minimal wrinkling and need for user manipulation. There is also a need for a flexible bioprocessing vessel that conforms to a cylindrical interior of a rigid support structure.

### Brief Description

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

According to an aspect of the invention, a flexible bioprocessing vessel includes at least six side panels forming sides of the flexible bioprocessing vessel, a top panel adjoining the at least six side panels, the top panel forming a top of the flexible bioprocessing vessel, a bottom panel adjoining the at least six side panels, the bottom panel forming a bottom of the flexible bioprocessing vessel, the at least six side panels, top panel, and bottom panel defining an interior cavity configured for processing a fluid, the at least six side panels joined together via vertical welds. The vessel further includes at least one fluid input and at least one fluid output for adding and removing fluid to and from the interior cavity respectively. Wherein the flexible bioprocessing vessel is configured for use within a rigid support structure having a cylindrical interior.

In an embodiment, a total number of the at least six side panels is selected based at least in part on a circumference of the cylindrical interior of the rigid support structure.

In an embodiment, the at least six side panels are seven side panels.

In an embodiment, the at least six side panels are eight side panels.

In an embodiment, the flexible bioprocessing vessel includes a plurality of tabs located proximate the top panel, the plurality of tabs allowing the flexible bioprocessing vessel to be lifted during deployment within the cylindrical interior of the rigid support structure.

In an embodiment, the plurality of tabs are six tabs circumferentially located about the top panel.

In an embodiment, the flexible bioprocessing vessel has a 500 L capacity.

In an embodiment, the flexible bioprocessing vessel has a 2000 L capacity.

In an embodiment, each of the at least six side panels are thermally welded to adjacent panels.

In an embodiment, each of the at least six side panels has a width that is less than its height.

In an embodiment, the flexible bioprocessing vessel has an overall height of about 1350 mm and an overall point to point width of about 814 mm and is configured for use with a 500 L rigid support structure having a cylindrical interior.

In an embodiment, the flexible bioprocessing vessel has an overall height of about 2100 mm and an overall point to point width of about 1304 mm and is configured for use with a 2000 L rigid support structure having a cylindrical interior.

In an embodiment, the flexible bioprocessing vessel may be folded for storage and/or transportation and unfolded for installation on a bottom surface of a rigid support structure having a cylindrical interior.

In an aspect of the invention, a system for bioprocessing includes a flexible bioprocessing vessel that features at least six side panels forming sides of the flexible bioprocessing vessel, a top panel adjoining the at least six side panels, the top panel forming a top of the flexible bioprocessing vessel, and a bottom panel adjoining the at least six side panels, the bottom panel forming a bottom of the flexible bioprocessing vessel, the at least six side panels, top panel, and bottom panel defining an interior cavity configured for processing a fluid. The system further includes a rigid support structure having a cylindrical interior that includes a bottom surface and a substantially open top.

In an embodiment, a total number of the at least six side panels is selected based at least in part on a circumference of the cylindrical interior of the rigid support structure.

In an embodiment, the at least six side panels may be seven side panels.

In an embodiment, the at least six side panels may be eight side panels.

In an embodiment, the flexible bioprocessing vessel includes a plurality of tabs located proximate the top panel, the plurality of tabs allowing the flexible bioprocessing vessel to be lifted during deployment within the rigid support structure.

In an embodiment, the plurality of tabs are six tabs circumferentially located about the top panel.

In an embodiment, the flexible bioprocessing vessel is a 500 L flexible bioprocessing vessel.

In an embodiment, the flexible bioprocessing vessel is a 2000 L flexible bioprocessing vessel.

In an embodiment, the rigid support structure includes a vessel hoist mechanism.

### BRIEF DESCRIPTION OF THE FIGURES

The present invention will be better understood from reading the following description of non-limiting embodiments, with reference to the attached drawings, wherein below:

FIG. 1 is a perspective view of a rigid support structure suitable for use with embodiments of the present invention;

FIG. 2 is an exploded perspective of a flexible bioprocessing vessel according to an embodiment of the invention illustrating individual panels for assembly;

FIG. 3 is an assembled perspective view of the flexible bioprocessing vessel of FIG. 2;

FIG. 4 is a top plan view of a flexible bioprocessing vessel deployed in a rigid support structure according to embodiments of the invention;

FIG. 5 is front plan view of the flexible bioprocessing vessel of FIG. 4;

FIG. 6 is a top perspective illustration of a flexible bioprocessing vessel in accordance with an embodiment of the invention showing the vessel in a deployed state within a rigid support structure;

FIG. 7 is a side perspective illustration of the flexible bioprocessing vessel and rigid support structure of FIG. 6; and

FIG. 8 is a perspective illustration of a flexible bioprocessing vessel and rigid baseplate according to an embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film.

A “vessel,” as the term is used herein, means a flexible bag, a flexible container, a semi-rigid container, or a rigid container, as the case may be. The term “vessel” as used herein is intended to encompass vessels, (e.g., bioprocessing vessels), having a wall or a portion of a wall that is flexible, single-use flexible bags, as well as other containers or conduits commonly used in biological or biochemical processing, including, for example, cell culture/purification systems, fermentation systems, mixing systems, media/buffer preparation systems, and filtration/purification systems.

As used herein, the term “bag” means a flexible or semi-rigid vessel used, for example, as a mixer or bioreactor for the contents within.

Embodiments may be utilized in connection with a wide variety of biological and chemical processes, which are referred to generally herein as “bioprocessing.” This term encompasses, but is not limited to, the various processes that occur in bioreactors, mixers, fermenters, and the like. A “bioprocessing vessel” is a vessel suitable for use with or in a bioreactor, mixer, fermenter, or other biological or chemical processing device. Certain embodiments may be suitable for use in other industries/applications where ease of installation, reduced vessel wrinkling, and/or versatile mixing of fluids is desirable.

Flexible vessels according to embodiments are not limited to specific working volumes. That is embodiments may have 500 L, 2000 L, 3000 L, and other volumes, and the invention is not limited in this regard. In certain embodiments, vessels may be as large as 6000 L.

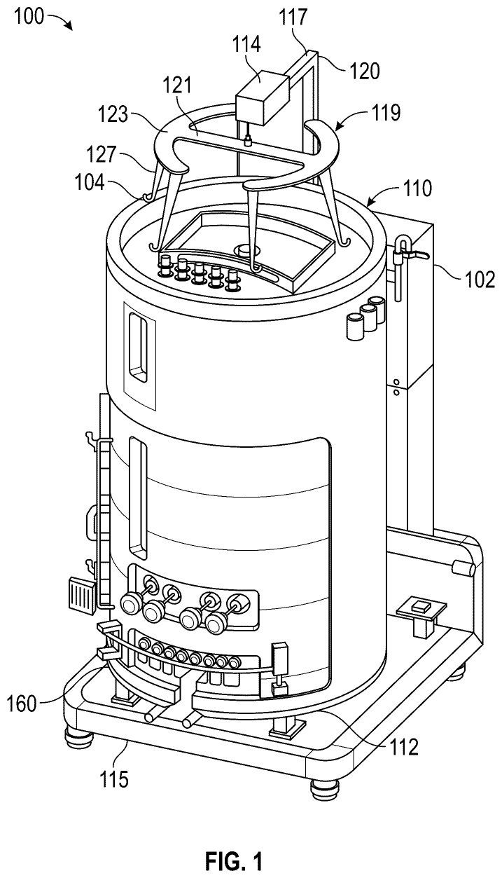

Referring now to FIG. 1, a rigid support structure 100 for supporting a flexible bioprocessing vessel according to an embodiment of the invention is depicted. The rigid support structure 100 includes a rigid body 102 having a cylindrical interior 104 that includes a bottom surface (not shown) and a cylindrical side surface that form the interior. The cylindrical interior 104 has a substantially open top 110 and a selectively openable door 112. As will be appreciated, the selectively openable door 112 allows access to the cylindrical interior 104. In certain embodiments, the cylindrical interior may have one or baffles to prevent/reduce a vortex effect during use (not shown).

In the depicted embodiment, the rigid body 102 has a cylindrical exterior which defines the cylindrical interior. As will be appreciated, however, in other embodiments the exterior of the rigid body 102 may have a shape, structure, or configuration that departs from the cylindrical interior 104. For example, in embodiments, the exterior may be a separate structure from the cylindrical interior, e.g., the rigid body may have a square, cuboid, cylindrical, or other shaped exterior while maintaining its cylindrical interior.

Referring again to FIG. 1, the selectively openable door 112 includes at least one opening 160 which facilitates connection of tubing, e.g., one or more fluid lines and/or probes (e.g., a sample line port, sensor ports, and drain ports) to a flexible bioprocessing vessel. That is, it allows tubing to exit the cylindrical interior. In a specific embodiment, the opening 160 allows access to a sample line port 309, sensor ports 313, and the drain port.

The rigid body 102 further includes a stand portion 115 attached to the rigid body 102 which allows for access to the space below the body. In the depicted embodiment, the stand portion 115 includes a plurality of legs. The number of legs may vary, however, and certain embodiments may utilize a structure other than legs to raise the rigid body 102 or otherwise allow access to an underside of the rigid body.

The depicted support structure further includes a vessel hoist mechanism 120 which includes a hoist motor 114 which is attached to an arm 117 that extends over the open top of the rigid body 102. The motor includes a selectively retractable line that is connected to an attachment frame 119 which, in an embodiment, includes a plurality of downwardly depending legs 127, which are configured for connection to a plurality of circumferentially arranged hoisting points arranged around the top of the flexible bioprocessing vessel. In the depicted embodiment, there are six legs, each leg being connected to a hoisting point on the vessel. In embodiments, there may be more than six circumferentially arranged hoisting points (and legs), e.g., seven or eight such points, or in some cases fewer than six.

In embodiments, the attachment frame 119 includes a central member 121 having opposed distal ends. The central member 121 may include an attachment point in substantially the center of the central member to which the selectively retractable line of the motor is attached. A curved or arcuate hoist member 123 is formed on or attached to each of the distal ends of the central member. Each hoist member has a radius of curvature that approximates or is complementary to a radius of curvature of the cylindrical support structure and/or the placement of circumferentially arranged hoisting points on the vessel. That said, in embodiments, the attachment frame 226 may be sized such that it may be lowered within the cylindrical interior of the rigid support structure. Each hoist member includes three legs that are configured for attachment to hoisting points on a vessel. As will be appreciated, other hoisting mechanisms may be usable and the embodiments of the invention are not limited to a specific mechanism.

As mentioned, rigid support structures having cylindrical interiors such as that depicted in FIG. 1 are typically used with cylindrical or semi-cylindrical flexible vessels that are manufactured from a relatively small number of flexible panels, e.g., three or four such panels, that are thermally welded together. Despite having relatively few panels, such vessels require a large number of welds to manufacture and are generally oversized, e.g., dimensionally larger than necessary to accommodate the maximum fill levels of the vessel. Moreover, the panel configuration and weld location results in vessels that are folded for shipment in a manner that is relatively space inefficient. Such vessels may also have a headspace that is not uniform in curvature or size.

These vessels are also deployed within a rigid support structure via inflation with air and one or more lift mechanisms that attach to the vessel and guide/raise it into place during inflation. Known vessels, however, have relatively few attachment points to be lifted from and given the number and shape of the panels in their construction, may not conform closely to a cylindrical interior of the rigid support structure. This results in the flexible panels (e.g., side panels) potentially sagging and wrinkling which is undesirable. Such wrinkling may necessitate user manipulation, e.g., a user pulls on the sides of the bag, to reduce/remove wrinkles.

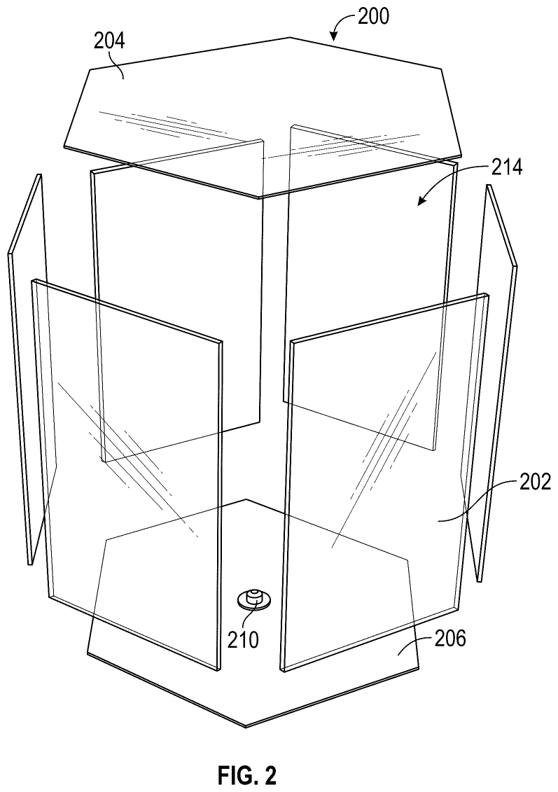

Referring now FIG. 2, a flexible bioprocessing vessel 200 according to an embodiment of the invention is depicted. As shown, the flexible bioprocessing vessel 200 includes six side panels 202 that are secured to a top panel 204 (which, in embodiments, may be a multi-panel design) and a bottom panel 206 that define an interior cavity 214 configured for processing a fluid.

The interior cavity 214 includes an impeller 210 (here the impeller seat, which is welded to the vessel, is shown without blades). In embodiments, the top panel 204 may include a protective cap (not shown) that covers and protects the impeller 210 during shipping/transport of the vessel.

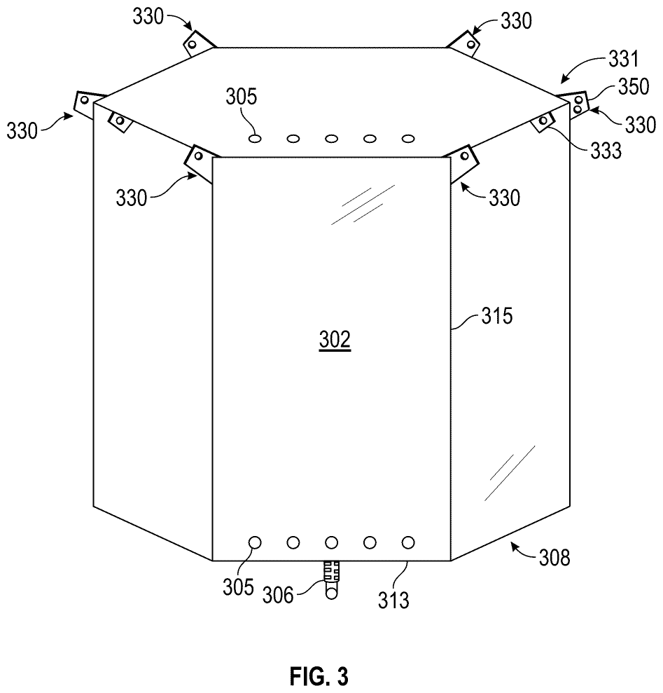

In embodiments, the six side panels, top panel and bottom panel are each thermally welded to adjacent panels. That is, the panels are joined together at seams 315, 317 (FIG. 3). As shown, there are vertical seams 315 that join side panels together as well as substantially horizontal seams 317 that join top and bottom panels to the side panels.

As will be appreciated, a variety of manufacturing techniques and processes may be employed to assemble, weld, or otherwise construct the inventive flexible bioprocessing vessel and the embodiments are not limited in this regard. Although depicted as being constructed from eight separate panels that include separate bottom and top panels, it may be possible for the bottom and/or top to be integrated into the six side panels that form the sides of the vessel.

Embodiments may provide an ease of manufacture compared to some existing three panel vessel designs. More specifically, embodiments may require a fewer number of seals, e.g., 18 seals, compared some existing vessels, which, despite having fewer panels, may require a relatively large number of seals or the sealing of a greater number of material plies, (e.g., 4-ply sealing). As will be appreciated. the fewer number of seals, the less labor and machine costs are involved.

In embodiments, additional panels may be added or removed to create larger or smaller vessels. That is, in embodiments, additional side panels may be incorporated to create wider/larger volume vessels. Certain embodiments may include seven, eight or more side panels. Indeed, a total number of side panels may be selected based at least in part on a circumference of the cylindrical interior of the rigid support structure,

As will be appreciated, embodiments of the invention provide a vessel with an exterior that more closely fits within a cylindrical support structure/tank than vessels that include three or four panels with substantially triangular or square cross sections. A close fit within the support structure reduces the possibility of vessel wrinkling during installation.

Referring now to FIG. 3, an assembled flexible bioprocessing vessel 300 configured for use with embodiments of the invention is depicted. The vessel 300 includes the aforementioned six flexible side panels 302 which form the sides of the flexible bioprocessing vessel. The flexible bioprocessing vessel 300 includes a flexible top panel 304 adjoining the six side panels 302 and forming a top of the flexible bioprocessing vessel 300, and a flexible bottom panel 308 located on an opposite end of the flexible bioprocessing vessel 300 from the top panel 304, the bottom panel 308 adjoining the six side panels 302 and forming a bottom of the flexible bioprocessing vessel 300. The six side panels 302, top panel 304, and bottom panel 308 define the interior cavity that holds fluid for mixing/processing.

The flexible bioprocessing vessel may further include fluid inputs/connectors/ports 305 for exhaust filtering, sensing, and adding fluids to the interior cavity. In embodiments, the fluid inputs/connectors/ports are located in one or more rows on a front facing flexible panel of the vessel, and on the top panel. As will be appreciated, the vessel includes at least one fluid input for fluid additions and at least one fluid output 306 for fluid removal. In certain embodiments, the inputs/connectors/ports are located on plates that are welded to the vessel.

Referring to FIG. 3, the vessel includes a plurality of circumferentially arranged hoisting points 330. The hoisting points 330 are located proximate to a junction 331 of each vertical seam and the horizontal seam of the top panel and side panels. The hoisting points may be a section of flexible film that is welded to the vertical and/or horizontal seam of the vessel. In embodiments, the hoisting points are film eyelet tabs 350 that include one or more eyelets. The eyelets may be formed as holes in the flexible film. In embodiments, the eyelets may be reinforced with grommets, which may be metal or plastic. The eyelets may be a variety of sizes and are not limited in this regard. Each film eyelet tab 350 may include multiple eyelets allowing for flexibility in the use of various hoisting mechanisms.

In certain embodiments, the circumferential hoisting points may also include tabs 333 which are adjacent to the junctions 331 and located on the horizontal seams. Certain embodiments may include both film eyelet tabs 350 and tabs 333, that is they may include hoisting points in a variety of locations for use with, for example, various hoisting mechanisms.

In embodiments there are six hoisting points located proximate to each of the seam junctions. In other embodiments, there may be more than six hoisting points, for example. a six-sided vessel may have eight or more hoisting points. In certain embodiments, the boisting points are evenly spaced about the circumference of the top panel of the vessel.

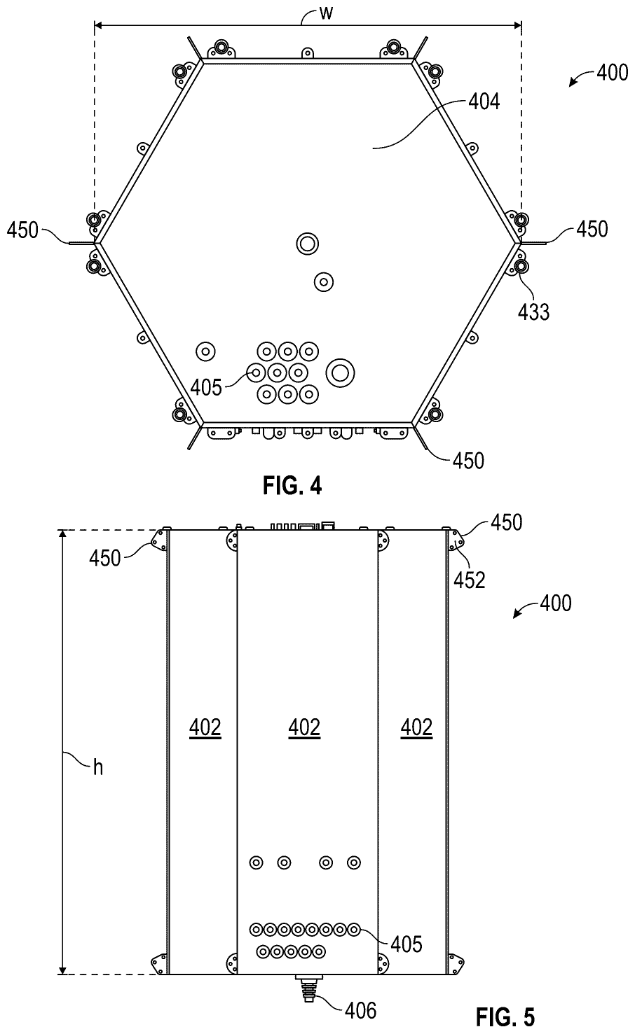

Referring now to FIGS. 4 and 5, a 2000 L flexible bioprocessing vessel according to an embodiment is depicted. The vessel 400 has a top panel 404 with a plurality of inputs/connectors/ports 405 and a six side panels 402. The vessel has an overall height h of about 2100 mm and a point-to-point overall width w of about 1304 mm. As shown, the embodiment includes circumferential hoisting points that are film eyelet tabs 450 that include one or more eyelets 452. The top panel further includes tabs 433 which further include holes/eyelets. As will be appreciated, it may be possible to connect some or all of the film eyelet tabs 450 and/or the tabs 433 to a hoist mechanism such those discussed herein.

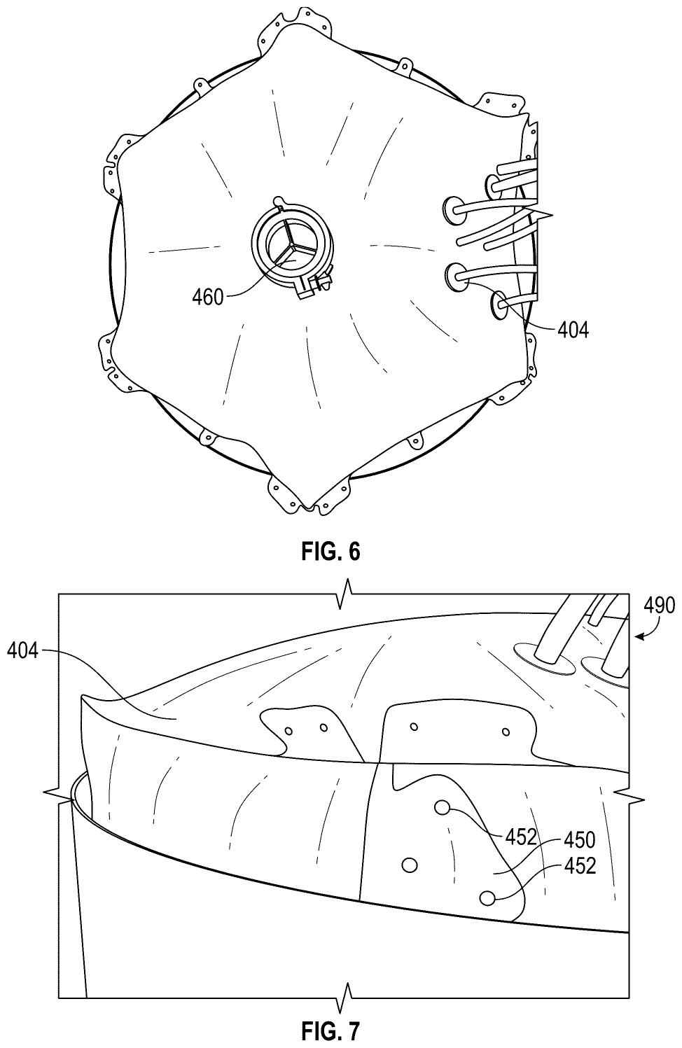

The circumferential hoisting points allow the side panels of the bag to be guided/pulled up during deployment which minimizes the presence of wrinkles. In addition to the circumferential hoisting point, embodiments may include a central attachment point 460 (FIG. 6).

In embodiments, a flexible bioprocessing vessel has a 500 L capacity. This flexible bioprocessing vessel has an overall height of about 1350 mm and an overall point to point width of about 814 mm.

In other embodiments, a flexible bioprocessing vessel has a 2000 L capacity. This vessel has an overall height of about 2100 mm and an overall point to point width of about 1304 mm.

Referring now to FIG. 7, in embodiments, the vessel 400 has a headspace 490 that, when the vessel is deployed and in use, is generally dome shaped and has a uniform curvature. This provides a large available area for fluid addition and flexibility in exhaust placement.

As will be appreciated, embodiments of the vessel may be manufactured from a variety of flexible materials, including various polymeric materials. The invention is not limited to any specific film material, thickness, or the like. The film may be a multi-layer composite or a single layer of material and may be opaque or transparent.

In embodiments, the vessel may be manufactured from a USP Class VI certified material such as, for example, silicone, polycarbonate, polyethylene, and polypropylene. Non-limiting examples of flexible materials include polymers such as polyethylene (for example, linear low-density polyethylene and ultra-low-density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. In an embodiment, the flexible material may be a laminate of several different materials.

While the six side panels are depicted as being equal in size, in certain embodiments, the panels may vary in size, e.g., width. That is, for example, two opposing panels may be of a first width and the remaining panels may be a second width greater or smaller than the first width. Likewise, the six side panels need not be unitary and each panel could be composed of two or more joined/welded subpanels, similar to the top panel. In certain embodiments. each side panel has a width that is less than its height.

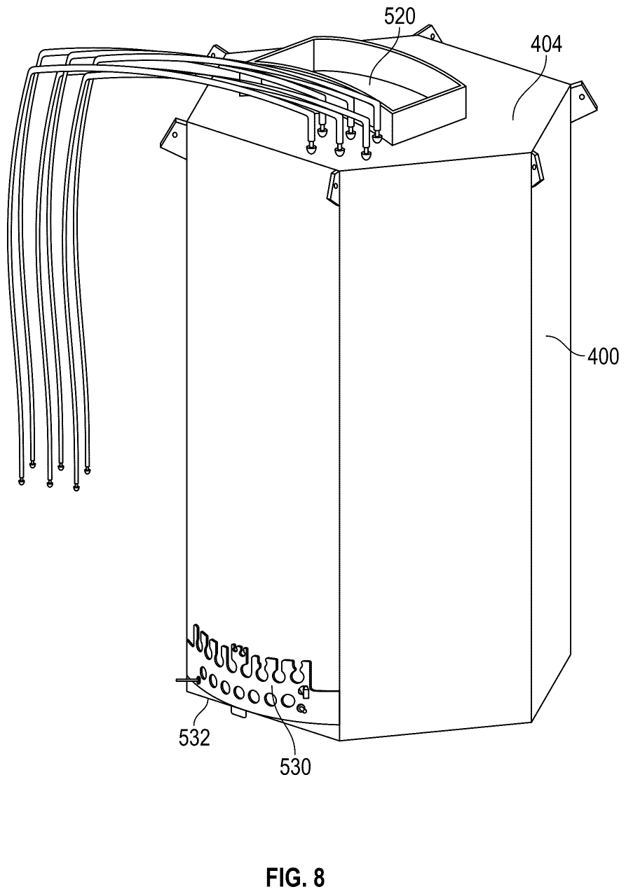

Referring now to FIG. 8, an embodiment of a 2000 L flexible bioprocessing vessel 400 is depicted. As shown, the vessel 400 is operatively connected to an apparatus that facilitates transportation/installation of the vessel 400. The apparatus includes a base portion 502. The base portion 502 is configured for operative connection to a bottom panel of the vessel, e.g., via direct attachment to the film of the bottom panel or to a vessel port or other component that is welded to the bottom panel. In embodiments, base portion has at least one handle allowing the vessel to be easily removed from external packaging and lifted into a rigid support structure.

As depicted, the base portion 502 also includes a front panel 530. In embodiments, the front panel 530 in curved and includes a plurality of apertures 532 configured to secure and protect tubing, connectors, and the like, extending from the flexible bioprocessing vessel during transportation and/or use. The front panel 530 may be removably attached to the base portion using a variety of fasteners.

The apparatus further includes a top plate 520 that is configured for operative connection to a top panel 402 of the flexible bioprocessing vessel 400, e.g., via direct attachment to the film of the top panel or to a vessel port or other component that is welded to the top panel where it remains during use of the vessel 200 for bioprocessing. In certain embodiments, the top plate may sit on the top panel with no direct structural attachment. The top plate 320 includes at least one slot or other features configured to receive and support tubing (e.g., fluid lines) and connectors (e.g., ports or other fittings) facilitating use of the flexible bioprocessing vessel.

In use, the vessel with the base portion and top plate are placed within a rigid support structure. The base portion and top plate are separated or disconnected, and the vessel may be deployed. The base portion and top plate remain operatively connected to the vessel during use and may also facilitate removal of the used vessel.

Embodiments of the invention also contemplate a method of folding a flexible bioprocessing vessel for storage and transportation, such as within an apparatus for transporting and installing the vessel. In a specific embodiment, the method involves placing the top panel flat on top of the bottom panel making sure that the impeller cover is positioned over the impeller to protect the vessel film. Then, the six corners of the top panel are pushed down onto the six corners of the bottom panel. At this stage, the six side panels that form the sides are substantially upright in a standing position.

Then, one by one, the panels on each side of a weld/seam between the six side panels are pushed together and each weld is folded over to an operator's left onto the top panel. This is repeated six times so the formerly upright sides are folded over onto the top panel. The resulting folded vessel now has a substantially hexagonal shape. At this point, the vessel may be folded again wherein opposing sides are folded together towards a center of the vessel with the fluid outlet (e.g., port) facing upward toward the operator. The vessel may now be placed into exterior packaging for shipment/transport. The vessel may then be unfolded for installation on a bottom surface of a rigid support structure.

As will be appreciated, despite the above, embodiments are not limited to a specific folding or packaging process or technique.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

While the dimensions and types of materials described herein are intended to define the parameters of the invention, they are by no means limiting and are exemplary embodiments. Many other embodiments will be apparent to those of skill in the art upon reviewing the above description.

The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

Moreover, in the following claims, terms such as “first,” “second,” “upper,” “lower,” “bottom,” “top,” etc. are used merely as labels, and are not intended to impose numerical or positional requirements on their objects. Further, the limitations of the following claims are not written in means-plus-function format and are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal language of the claims.

Claims:

1. A flexible bioprocessing vessel comprising:
at least six side panels forming sides of the flexible bioprocessing vessel;
a top panel adjoining the at least six side panels, the top panel forming a top of the flexible bioprocessing vessel;
a bottom panel adjoining the at least six side panels, the bottom panel forming a bottom of the flexible bioprocessing vessel, the at least six side panels, top panel, and bottom panel defining an interior cavity configured for processing a fluid, the at least six side panels joined together via vertical welds; and
at least one fluid input and at least one fluid output for adding and removing fluid to and from the interior cavity respectively;
wherein the flexible bioprocessing vessel is configured for use within a rigid support structure having a cylindrical interior.

2. The flexible bioprocessing vessel of claim 1 wherein a total number of the at least six side panels is selected based at least in part on a circumference of the cylindrical interior of the rigid support structure.

3. The flexible bioprocessing vessel of claim 1 wherein the at least six side panels are seven side panels.

4. The flexible bioprocessing vessel of claim 1 wherein the at least six side panels are eight side panels.

5. The flexible bioprocessing vessel of claim 1 wherein the flexible bioprocessing vessel includes a plurality of tabs located proximate the top panel, the plurality of tabs allowing the flexible bioprocessing vessel to be lifted during deployment within the cylindrical interior of the rigid support structure.

6. The flexible bioprocessing vessel of claim 1 wherein the flexible bioprocessing vessel has a 500 L capacity.

7. The flexible bioprocessing vessel of claim 1 wherein the flexible bioprocessing vessel has a 2000 L capacity.

8. The flexible bioprocessing vessel of claim 1 wherein each of the at least six side panels are thermally welded to adjacent panels.

9. The flexible bioprocessing vessel of claim 1 wherein each of the at least six side panels has a width that is less than its height.

10. The flexible bioprocessing vessel of claim 1 wherein the flexible bioprocessing vessel has an overall height 2100 mm and an overall width of about 1304 mm and is configured for use with a 2000 L rigid support structure having a cylindrical interior.

11. The flexible bioprocessing vessel of claim 1 wherein the flexible bioprocessing vessel may be folded for storage and/or transportation and unfolded for installation on a bottom surface of a rigid support structure having a cylindrical interior.

12. A system for bioprocessing comprising:
a flexible bioprocessing vessel comprising:
at least six side panels forming sides of the flexible bioprocessing vessel;
a top panel adjoining the at least six side panels, the top panel forming a top of the flexible bioprocessing vessel;
a bottom panel adjoining the at least six side panels, the bottom panel forming a bottom of the flexible bioprocessing vessel, the at least six side panels, top panel, and bottom panel defining an interior cavity configured for processing a fluid; and
a rigid support structure having a cylindrical interior that includes a bottom surface and a substantially open top.

13. The system of claim 12 wherein a total number of the at least six side panels is selected based at least in part on a circumference of the cylindrical interior of the rigid support structure.

14. The system of claim 12 wherein the at least six side panels are seven side panels.

15. The system of claim 12 wherein the at least six side panels are eight side panels.

16. The system of claim 12 wherein the flexible bioprocessing vessel includes a plurality of tabs located proximate the top panel, the plurality of tabs allowing the flexible bioprocessing vessel to be lifted during deployment within the rigid support structure.

17. The system of claim 16 wherein the plurality of tabs are six tabs circumferentially located about the top panel.

18. The system of claim 12 wherein the flexible bioprocessing vessel is a 500 L flexible bioprocessing vessel.

19. The system of claim 12 wherein the flexible bioprocessing vessel is a 2000 L flexible bioprocessing vessel.

20. The system of claim 12 wherein the rigid support structure includes a vessel hoist mechanism.
